(19) United States
(12) Plant Patent Application Publication
(10) Pub. No.: US 2015/0237784 P1
(43) Pub. Date: Aug. 20, 2015

(54) Mandarin variety named 'BELALATE'

(50) ;

(71) Applicant: Jose Beltran Ano, Guadassuar (ES)

(72) Inventor: Jose Beltran Ano, Guadassuar (ES)

(21) Appl. No.: 13/999,398

(22) Filed: Feb. 20, 2014

(51) Int. Cl. A01H 5/00

(52) U.S. Cl. PLT/202

(57) ABSTRACT

A new and distinct variety of Citrus reticulata called ‘Belalate’ is distinguished by the production of fruit that combines late season maturity, medium fruit size, an orange-colored rind with a very smooth texture, and a sweet flavor. It further distinguishes itself by being seedless and easy to peel.

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Citrus reticulata.

### VARIETY DENOMINATION

The variety denomination is ‘Belalate’.

### BACKGROUND OF THE INVENTION

The present invention relates to a new and distinct variety of Citrus reticulata and herein referred to by its cultivar name ‘Belalate’ which distinguishes itself by producing a medium, orange-colored seedless fruit, a very smooth rind texture, and a sweet flavor, that matures between the first week of January and end of February when grown in Guadassuar Valencia, Spain. The new, variety was first discovered by Jose Beltran Ano in 2005. It originated as a spontaneous mutation of an ‘Owari Satsuma’ (unpatented) mandarin that was growing in a cultivated orchard near Guadassuar, Valencia. Thereafter, the new variety was successfully asexually reproduced in 2008 in Guadassuar, Valencia, Spain by grafting budwood onto ‘Carrizo’ citrange rootstock. Subsequent grafting on ‘Carrizo’ citrange and ‘Cleopatra’ mandarin rootstocks in Guadassuar, Valencia, Spain has shown the features of the new variety to be stable and reproduce true to type in successive propagations. In 2008,budwood of the new variety was delivered to the Instituto Valenciano de Investigaciones Agrarias (the “IVIA”) in order to clean the variety of all know viruses. In 2010, the IVIA delivered to the owner certified clean material to be reproduced in a nursery. After several additional asexual propagations in a nursery located in Tarragona, Spain, the new variety was planted in a citrus block located in Carcer, Valencia, Spain.

### SUMMARY OF THE INVENTION

‘Belalate’ is characterized by producing a medium, orange-colored seedless fruit that is mature for harvesting and shipment between approximately the first week of January and the end of February when grown in Guadassuar, Valencia, Spain. The new variety can be compared to ‘Owari Satsuma’ (unpatented) mandarin but the fruit produced by the new variety mature approximately two months later, among other distinguishing characteristics.

### BRIEF DESCRIPTION OF THE DRAWINGS

The accompanying photographic drawings illustrate the new variety with the colors being as true as possible with color illustrations of this type. The photographs were taken under natural light conditions.

FIG. 1 illustrates a five-year-old tree of ‘Belalate’;

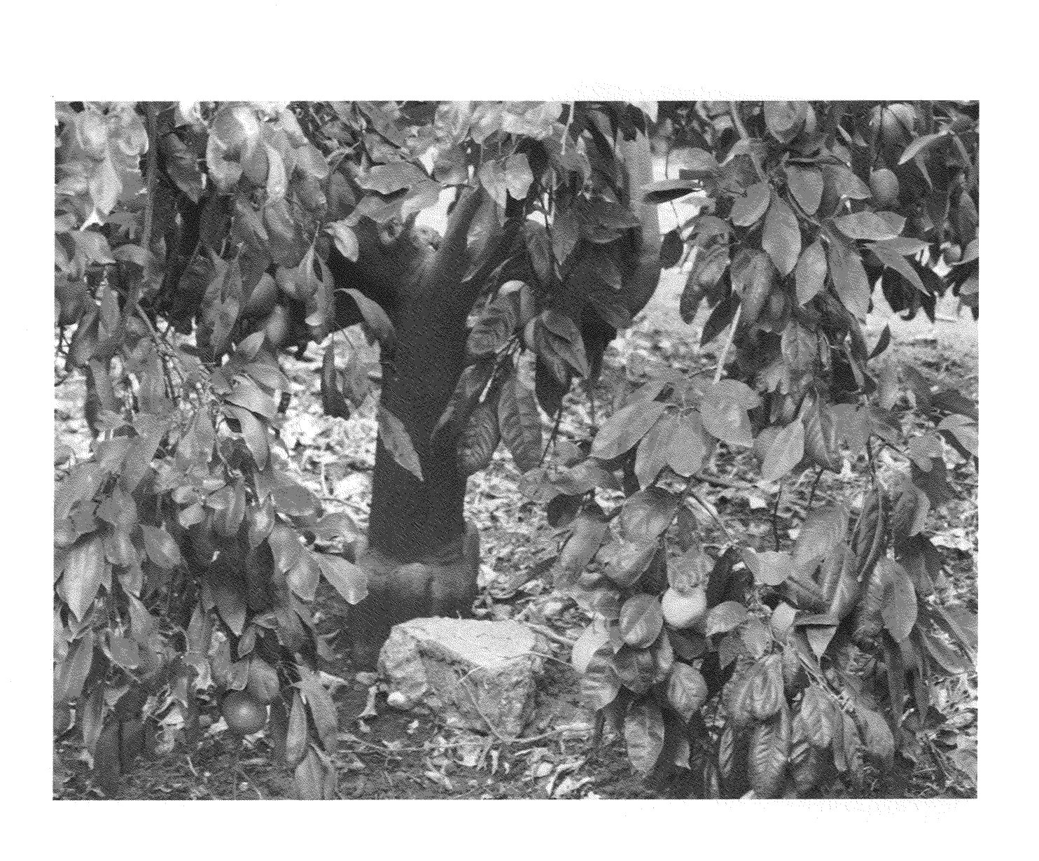

FIG. 2 illustrates a leaf of ‘Belalate’; and

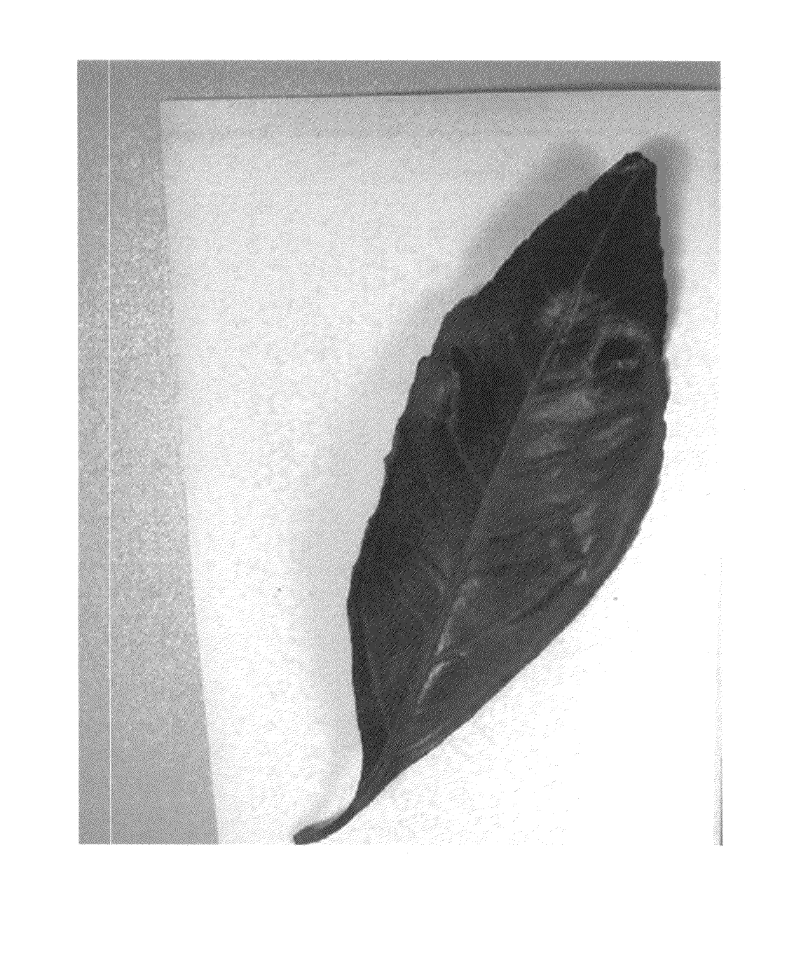

FIG. 3 illustrates fruit from a five-year-old tree of ‘Belalate’.

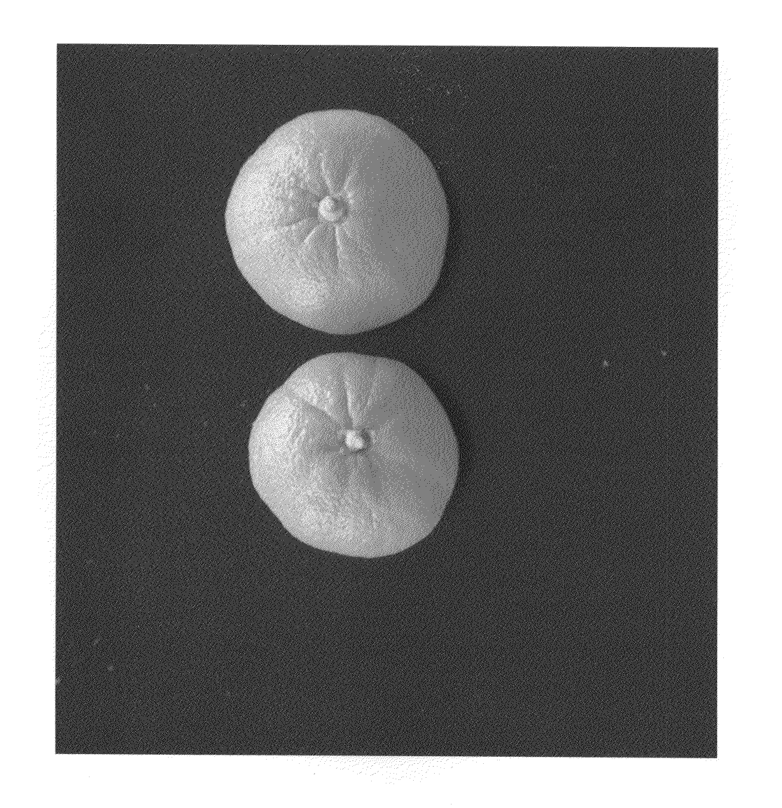

### DETAILED BOTANICAL DESCRIPTION

Referring more to the horticultural description of the new and distinct variety, the following has been observed under the ecological conditions prevailing at the origin orchard located near Guadassuar, Valencia, Spain. The plant history was taken on a five (5) year old tree. All major color descriptions are by reference to the R.H.S. Color Chart of The Royal Horticultural Society of London. Common colors names are also used in several instances.
Tree: The growth rate is vigorous. Tree growth habit is drooping, without spines. The height of the five (5) year old tree is approximately 3.0 meters, and the spread, depending on spacing, is approximately 3.0 meters. The trunk diameter is approximately 50 to 60 cm measured approximately 25 cm above the soil line, and has a globose structure. Belalate adapts to almost all types of soil except for highly saline and calcareous soil.
Leaf: Leaves of ‘Belalate’ are long, with a length of approximately 80 to 90 mm and a width of approximately 40 to 50 mm. The leaves are straight or weakly concave in cross section, with an acuminate apex. Twisting and blistering are absent. The leaves are medium green in color, with crenate incisions but without undulations of margins in the leaf blade. Petioles are long and normal lacking wings. Leaves are large, lanceolate and coriaceous, with very pronounced veins on both the top and the underside surfaces.
Flowers: Flowering of ‘Belalate’ occurs beginning at the end of March and continues through April in Guadassuar, Valencia, Spain. Flowers are hermaphroditic with five (5) white colored petals and light yellow colored anthers. A petal is 12 to 14 mm long, and 6 to 8 mm in width. The length of stamens is medium, the diameter of calyx is medium, and the style is long. The peduncle is very thick, approximately twice the width of ‘Owari Satsuma’ (unpatented).
Fruit: Fruit of ‘Belalate’ is mature between the first week of January to the end of February in Guadassuar Valencia, Spain. The fruit is flat in shape with no neck. The fruit has a concave distal end of medium depth and small diameter. The fruit is medium sized for a mandarin averaging 60 mm in diameter and 45 mm in height with a very smooth, medium orange rind color (RHS Orange N12A) and large, conspicuous oil glands. The weight of the fruit is 90 to 110 grams. There is no presence of constriction at stalk end. There are a number of short radial grooves the broadest of which are positioned at the middle. There is no presence of collar. Fruit has an incomplete smooth areola with medium diameter.
Rind: The surface texture of the rind of ‘Belalate’ is rough and is medium orange colored. Glossiness is absent or very weak. The rind is slightly adherent at maturity (easy to peel) and thin between 1.8 to 2.4 mm in thickness. The color of Albedo is white with loose density and large amount of strands.
Flesh: The fruit interior has a fine flesh texture with 9 to 11 segments and a semi-hollow axis of medium size at maturity. There is no indication of puffing. The predominate color of the flesh is medium orange. The filling of core is medium, with a medium diameter. The neck is not present. Presence of rudimentary medium strength segments is absent or weak.
Juice: The fruit of ‘Belalate’ are juicy between 48% to 53% juice. The length of juice vesicles is short and has a medium thickness and coherence. The content of total soluble solids is low. The soluble solids is 9 to 12 Brix. The % acid is 0.9 to 1.1. The juice color is orange.
Seeds: Fruit of ‘Belalate’ is seedless. The new variety is parthenocarpic; plants are self-incompatible.
Fruit holding ability on tree past maturity: The fruit holding ability on tree past maturity is approximately two (2) weeks.
Use: The use of ‘Belalate’ is fresh market.
Fruit quality after storage: Excellent, 4 to 6 weeks at 6° C.
Resistance to disease/pests: ‘Belalate’ is resistant to the citrus leafminer.

1. A new and distinct variety of Citrus reticulata, substantially as described and illustrated herein.
